(19) United States
(12) Patent Application Publication
(10) Pub. No.: US 2026/0274786 A1
(43) Pub. Date: Sep. 17, 2026

(54) PROCESS FOR PREPARING AZINE USING REACTORS IN CASCADE

(71) Applicant: ARKEMA FRANCE, Colombes (FR)

(72) Inventors: Jean-Marc SAGE, Colombes (FR); Valérie HENRARD, Colombes (FR)

(21) Appl. No.: 18/875,682

(22) PCT Filed: Jun. 23, 2023
(86) PCT No.: PCT/FR2023/050950

§ 371 Date: Dec. 16, 2024

(30) Foreign Application Priority Data
Jun. 24, 2022 (FR) FR2206297

(51) Int. Cl. C07C 249/16; B01J 19/18; C07C 241/02

(52) U.S. Cl. CPC C07C 249/16; B01J 19/1862; C07C 241/02

(57) ABSTRACT

The invention relates to a process for continuously preparing azine, comprising a step of reacting ammonia, hydrogen peroxide and a ketone of formula R₁R₂CO, the groups R₁ and R₂ denoting, independently of one another, a methyl, ethyl, propyl, butyl, pentyl, hexyl, heptyl and octyl group, in the presence of an aqueous solution comprising at least one activator to form an azine, the reaction being performed in at least 2 reactors arranged in cascade, the agitation of the first reactor being less than the agitation of the following reactor or each of the following reactors, the reactants being injected at the first reactor, the aqueous solution comprising at least one activator and comprising dissolved ammonia in a proportion of between 50% and 100% relative to the saturation of ammonia in pure water at the temperature of the aqueous solution.

The present invention relates to a process for preparing azine.

The present invention more specifically relates to a process for preparing azine obtained in the presence of a ketone by oxidation of ammonia with hydrogen peroxide and in the presence of an activator.

Hydrazine is employed in a variety of different applications, primarily in the deoxygenation of boiler waters (of nuclear power stations, for example), and is used for preparing pharmaceutical and agrochemical derivatives.

There is therefore an industrial need for the preparation of hydrazine hydrate, obtained from the hydrolysis of the azine.

Hydrazine hydrate is produced industrially by the Raschig or Bayer processes or from hydrogen peroxide.

In the Raschig process, ammonia is oxidized with a hypochlorite to give a dilute solution of hydrazine hydrate, which must then be concentrated by distillation. This process, being relatively unselective, relatively unproductive and highly polluting, is virtually no longer used.

The Bayer process is an improvement on the Raschig process, which involves shifting a chemical equilibrium by using acetone to trap the hydrazine formed in the form of azine of the following formula:

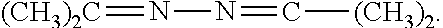

The azine is subsequently isolated and then hydrolysed to hydrazine hydrate. Yields are improved, but there is no improvement in environmental emissions.

The hydrogen peroxide process involves oxidizing a mixture of ammonia and a ketone with hydrogen peroxide in the presence of a means for activating the hydrogen peroxide to make the azine directly, which then only needs to be hydrolysed to hydrazine hydrate. The yields are high and the process is less polluting. This hydrogen peroxide process is described in numerous patents, for example U.S. Pat. Nos. 3,972,878, 3,972,876 and 4,093,656.

These processes are also described in Ullmann's Encyclopedia of Industrial Chemistry (1989), vol. A 13, pages 180-183 and the references included.

In the hydrogen peroxide processes, the ammonia is oxidized with the hydrogen peroxide in the presence of a ketone and a means for activating the hydrogen peroxide according to the following overall reaction, forming an azine:

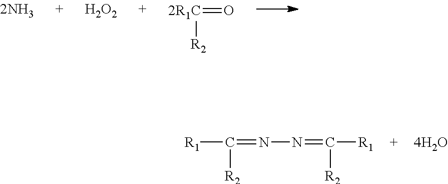

The activation means, or activator, may be a nitrile, an amide, a carboxylic acid or else a selenium, antimony or arsenic derivative. The azine is then hydrolysed to hydrazine and the ketone is regenerated according to the following reaction:

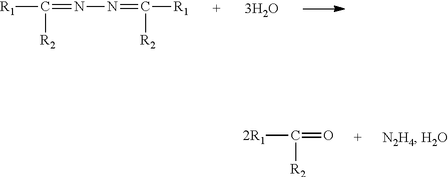

This hydrolysis is actually performed in two stages, with formation of an intermediate hydrazone:

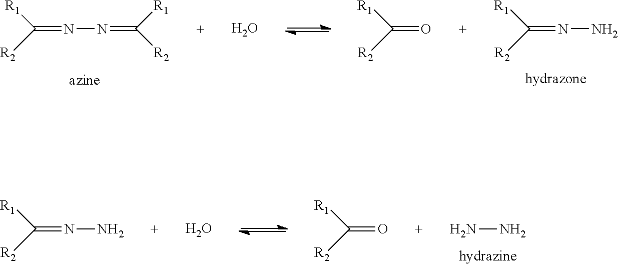

Whether the azine is produced by a hydrogen peroxide process or another process, methyl ethyl ketone is advantageously used because it is poorly soluble in an aqueous medium.

Specifically, in the hydrogen peroxide process, the azine of methyl ethyl ketone is relatively insoluble in the reaction medium, which is necessarily aqueous owing to the use of commercial aqueous solutions of hydrogen peroxide with a concentration of between 30% and 70% by weight. This azine can therefore be easily recovered and separated by simple decanting. It is highly stable particularly in an alkaline medium, i.e. in the ammoniacal reaction medium. In the current processes, this azine is subsequently purified, and then hydrolysed in a reactive distillation column to finally release methyl ethyl ketone at the top to be recycled, and above all an aqueous solution of hydrazine hydrate at the bottom. This must contain as few carbon products as possible as impurities and must be colourless.

A process for efficiently preparing hydrazine hydrate is known from document WO 2020/229773.

However, improvements to this process are still sought. In other words, an increase in the degree of conversion of the reactants, an increase in the final yield and a reduction in secondary reactions are constantly sought, so that the production is as efficient as possible.

The reaction for forming the azine is relatively complex, since it involves three phases: a gaseous phase with ammonia, an organic phase with the ketone, and an aqueous phase with the activator and hydrogen peroxide. However, for the reaction to be efficient, it is necessary for the reactants to come into contact with one another. Thus, the yield of this reaction is directly linked to the exchanges and contacts between the phases of the reactants.

It is known from the scientific article ‘Agitation Effects in a Gas-Liquid-Liquid Reactor System: Methyl Ethyl Ketazine Production’ by R. Kaur and K. D. P. Nigam in the journal International Journal of Chemical Reactor Engineering, from January 2007, that the agitation is a decisive improvement factor. It was observed that the higher the agitation speed, the more the yield increases, up to a threshold value of 600 rpm. However, these experiments were carried out in a semi-batch reactor. Yet, in general, industrial sites are equipped with continuous processes. Moreover, agitation of 600 rpm applied to industrial-volume reactors represents a significant consumption of energy. Consequently, solutions are still sought in order to make these gas-liquid-liquid contacts efficient, whether in terms of yield or energy consumption, within a continuous industrial process.

Specifically, the inventors have discovered that by carrying out the mixing reaction in several reactors and by adapting the respective agitation of the reactors, they were able to reduce the formation of unwanted byproducts of the reaction. In particular, it has been found that the process according to the invention makes it possible to reduce the production of aminoperoxide, a byproduct of the reaction. Aminoperoxide is described in application FR 2950887, for example. This reduction is observed across all of the reactors.

### BRIEF DESCRIPTION OF THE FIGURES

FIG. 1 is a diagram of the device implementing the claimed process.

FIG. 2 is a diagram of the device implementing the claimed process according to another embodiment.

### BRIEF DESCRIPTION OF THE INVENTION

Thus, a subject of the present invention is a process for continuously preparing azine, comprising a step a) of reacting ammonia, hydrogen peroxide and a ketone of formula R₁R₂CO, the groups R₁ and R₂ denoting, independently of one another, a methyl, ethyl, propyl, butyl, pentyl, hexyl, heptyl and octyl group, in the presence of an aqueous solution comprising at least one activator to form an azine,
the reaction being performed in at least 2 reactors arranged in cascade,
the agitation of the first reactor being less than the agitation of the following reactor or each of the following reactors,
the hydrogen peroxide, the ketone and an aqueous solution comprising at least one activator and comprising dissolved ammonia, preferably in a proportion of between 50% and 100% relative to the saturation of ammonia in pure water at the temperature of the aqueous solution, being injected into the first reactor.

### DETAILED DESCRIPTION OF THE INVENTION

Other features, aspects, subjects and advantages of the present invention will become even more clearly apparent from reading the description that follows.

It is specified that the expressions “from . . . to . . . ” and “of between . . . and . . . ” used in the present description should be understood as including each of the limits mentioned.

For the purposes of the present invention, “continuous” is understood to mean that the stream of the reactants introduced into the reactor and of the products synthesized during the process is uninterrupted.

The preparation of hydrazine hydrate is carried out according to the following steps:
ammonia, hydrogen peroxide and an alkyl ketone of formula R₁R₂C═O are reacted in the presence of an aqueous solution comprising at least one activator to form an azine,
the azine of the ketone formed is hydrolysed to obtain hydrazine hydrate.

The invention relates to the first step of this process.

### The Reactants

### Hydrogen Peroxide

Hydrogen peroxide may be used in its common commercial form, for example in aqueous solution comprising between 30% and 90% by weight of oxygen peroxide.

Advantageously, it is possible to add one or more customary stabilizers for peroxide solutions, for example phosphoric, pyrophosphoric, citric, nitrilotriacetic or ethylenediaminetetraacetic acid or the ammonium or alkali metal salts of these acids.

It is also known practice to stabilize the hydrogen peroxide solutions by adding sequestering agents thereto, which will complex the metal ions. This inhibits the redox reaction of the hydrogen peroxide. Sequestering agents particularly used to stabilize the hydrogen peroxide solutions are compounds of the type comprising phosphonic functions, in their acid form or in their salt form. The following commercial products may be used:
the product sold under the name DEQUEST® 2060 by the company Monsanto, which is an aqueous solution of 50% diethylenetriamine penta(methylene phosphonic acid),
the product sold under the name DEQUEST® 2041, which is an aqueous solution of ethylenediamine tetra(methylene phosphonic acid),
the products sold under the names DEQUEST® 2010 and 2006, respectively an aqueous solution of 60% 1-hydroxyethylidene-1,1-diphosphonic acid and an aqueous solution of 29% aminotris(methylenephosphonic acid) and 40% pentasodium salt of this acid.

These acids may also be used in their acid form or else completely or partially neutralized form, for example in the form of the sodium salt or the ammonium salt.

The amount to be used is advantageously between 10 and 1000 ppm and preferably between 50 and 250 ppm of all of the reactants and the solution comprising at least one activator at the inlet of the reactor.

### The Alkyl Ketone

The alkyl ketone of formula R₁R₂CO comprises groups R₁ and R₂ denoting, independently of one another, a methyl, ethyl, propyl, butyl, pentyl, hexyl, heptyl and octyl group. Preferably, dimethyl ketone and methyl ethyl ketone are used. Particularly preferably, methyl ethyl ketone is used. Hence, the preferred azine is the azine of methyl ethyl ketone, called MEKazine.

### The Activator

“Activator” is understood to mean a compound enabling the activation of the hydrogen peroxide, i.e. a compound that enables the azine to be produced from ammonia, hydrogen peroxide and a ketone.

This activator may be selected from organic or inorganic oxyacids, ammonium salts thereof and derivatives thereof: anhydrides, esters, amides, nitriles, acyl peroxides, or mixtures thereof. Advantageously, use is made of amides, ammonium salts and nitriles.

By way of example, mention may be made of:
(i) amides of carboxylic acids of formula R₅COOH in which R₅ is hydrogen, a linear alkyl radical having from 1 to 20 carbon atoms, or a branched or cyclic alkyl radical having from 3 to 12 carbon atoms, or an unsubstituted or substituted phenyl radical, (ii) amides of polycarboxylic acids of formula R₆(COOH)ₙ in which R₆ represents an alkylene radical having from 1 to 10 carbon atoms and n is an integer greater than or equal to 2; R₆ may be a single bond, in which case n is 2.

The radicals R₅ and R₆ may be substituted by halogens or by OH, NO₂ or methoxy groups. Mention may also be made of the amides of organic acids of arsenic. Organic acids of arsenic are, for example, methylarsonic acid, phenylarsonic acid and cacodylic acid.

The preferred amides are formamide, acetamide, monochloroacetamide and propionamide, and more preferentially acetamide.

Among the ammonium salts, use is advantageously made of the salts of hydracids, of inorganic oxyacids, of arylsulfonic acids, of acids of formula R₅COOH or R₆(COOH)ₙ, where R₅, R₆ and n are as defined above, and of organic acids of arsenic.

The preferred ammonium salts are formate, acetate, monochloroacetate, propionate, phenylarsonate and cacodylate.

Among the nitriles, mention may advantageously be made of the products of formula R₇(CN)ₙ, where n may range from 1 to 5 depending on the valence of R₇, R₇ is a cyclic or noncyclic alkyl having from 1 to 12 carbon atoms or a benzyl or a pyridinyl group. R₇ may be substituted by groups which are not oxidized in the reactor of step (a), for example halogens or carboxyl, carboxylic ester, nitro, amine, hydroxyl or sulfonic acid groups.

The preferred nitriles are acetonitrile and propionitrile.

The solution comprising at least one activator is formed by dissolving one or more products selected from organic or inorganic oxyacids, ammonium salts thereof and derivatives thereof: anhydrides, esters, amides, nitriles, acyl peroxides, or mixtures thereof as defined above. Advantageously, use is made of the above nitriles, ammonium salts or amides. Particularly preferably, use is made of a single activator, which is acetamide.

This solution is aqueous. According to another embodiment, said solution is an aqueous solution of an amide of a weak acid and the ammonium salt corresponding to this acid, as described in patent EP 0 487 160.

These weak-acid amides are derivatives of the corresponding carboxylic acids that have a dissociation constant of less than 3×10⁻³, in other words acids that have a pKa of greater than 3 in aqueous solution at 25° C.

For polycarboxylic acids, the acids in question are those for which the first ionization constant is less than 3×10⁻³.

By way of example, mention may be made of carboxylic acids of formula R₈COOH in which R₈ is a linear alkyl radical having from 1 to 20 carbon atoms, or a branched or cyclic alkyl radical having from 3 to 12 carbon atoms, or an unsubstituted or substituted phenyl radical, and polycarboxylic acids of formula R₉(COOH)ₙ in which R₉ represents an alkylene radical having from 1 to 10 carbon atoms and n is a number greater than or equal to 2; R₉ may be a single bond, in which case n is 2. The radicals R₈ and R₉ may be substituted by halogens or by OH, NO₂ or methoxy groups. Preference is given to using acetamide, propionamide, n-butyramide or isobutyramide.

The corresponding ammonium salt of acetamide is ammonium acetate.

It would not be outside the scope of the invention to form the ammonium salt in situ, i.e. to use the corresponding carboxylic acid which, by reaction with ammonia, gives the ammonium salt.

The proportions of the amide and of the corresponding ammonium salt may vary within wide limits. Use is commonly made of 1 to 25 parts of the ammonium salt per 5 parts of amide, and preferably 2 to 10.

### Ammonia

Ammonia is dissolved in the aqueous solution, which comprises at least one activator.

The solubility of gaseous ammonia in pure water as a function of the temperature is known from the book Lange's Handbook of Chemistry, Editor John A. Dean, 12^{th} edition, 1979, on page 10.3. This solubility is expressed as weight of gas dissolved in 100 grams of water at a pressure of 760 mm of mercury. The table disclosed on page 10.3 is reproduced below:

TABLE 1

| Temperature (° C.) | Amount in grams |
|---|---|
| 20 | 52.9 |
| 24 | 48.2 |
| 28 | 44.0 |
| 30 | 41.0 |
| 40 | 31.6 |
| 50 | 23.5 |
| 60 | 16.8 |
| 70 | 11.1 |

These values express the maximum solubility of ammonia in pure water, i.e. the saturation of pure water by ammonia. In the context of the invention, an aqueous solution comprising dissolved ammonia, preferably in a proportion of between 50% and 100% relative to the saturation of ammonia in pure water at the temperature of the aqueous solution, and comprising at least one activator is introduced into the first reactor. This solubility of ammonia in the aqueous phase is expressed relative to the amount of water contained in the aqueous phase comprising at least one activator.

In other words, starting from the values that are disclosed in the aforementioned Lange's Handbook of Chemistry and reproduced in Table 1 above, at 20° C., a dissolution of ammonia of 26.45 g to 52.9 g of ammonia is targeted. At 70° C., a dissolution of 5.55 g to 11.1 g of ammonia is targeted.

Preferably, ammonia is dissolved in the aqueous solution containing activator in a proportion of between 50% and 85% relative to the saturation of ammonia in pure water at the temperature of the aqueous solution.

Preferably, the temperature of the aqueous solution is lower than that of the reactor. More particularly, the temperature of the aqueous solution is more than 10° C. lower than the temperature of the reactor, and more preferentially still more than 20° C. lower than the temperature of the reactor.

The reactants may be used in stoichiometric amounts. However, it is possible to use from 0.2 to 5 mol and preferably from 1.5 to 4 mol of ketone, and from 0.1 to 10 mol and preferably from 1.5 to 4 mol of ammonia, per mole of hydrogen peroxide. The amount of solution comprising at least one activator may be between 0.1 and 2 kg per mole of hydrogen peroxide. This amount depends on its quality, i.e. on its catalytic strength or its activity which enables conversion of the reactants to azine. The above-stipulated proportions of the reactants make it possible to obtain a maximum conversion, typically greater than 90%, preferably greater than 95%, of hydrogen peroxide and a production of azine corresponding to more than 75% of the hydrogen peroxide employed, and possibly reaching 90%.

### a) Mixing Reaction

The reaction may be performed within a very wide temperature range, for example between 0° C. and 100° C., and operation is advantageously between 30° C. and 70° C. It is possible for there to be a temperature gradient between the various reactors. For example, the temperature of the first reactor may be around 45° C., while that of the last reactor may be around 60° C. While operation is possible at any pressure, it is simpler to be at atmospheric pressure, although an increase up to approximately 10 bar absolute is possible. Preferably, the reaction is carried out at between 1 and 5 bar absolute.

### The Reactors

The reaction takes place in at least two reactors arranged in cascade. Preferably, 3, 4 or 5 reactors in cascade are used.

The agitation in the first reactor is less than the agitation of the second reactor, and of any following reactor(s).

For the purposes of the present invention, agitation is understood to mean the speed of the stream of the reaction medium within the reactor that is generated by the movement of a rotating element, such as a blade, a baffle, an anchor or any other rotating element, or by a Venturi effect. For a medium agitated by a rotating element, the agitation of the reaction medium can be expressed by the agitation speed of the rotating element itself.

When the process uses 3 reactors or more, the reactors positioned after the first reactor may be agitated at an identical speed. It is also possible for the reactors positioned after the first reactor to be agitated at an increasing speed, that is to say that the third reactor is agitated at a speed greater than the agitation speed of the second reactor.

The reactors may have an internal diameter of between 1 and 6 m, preferably between 2 and 5 m. The working height of the reactor may be between 1 and 10 m, preferably between 3 and 7 m. Thus, the reaction volume may be between 25 and 100 m³ preferably between 40 and 70 m³. The reactors may have the same volume or else a different volume.

Preferably, the reactors may be equipped with agitation means, for example blades.

Thus, the reactor may comprise several agitation stages, preferably two agitation stages. Each agitation stage may comprise several inclined blades. Preferably, the blades are positioned in the lower third of the reactor and in the upper third of the reactor. The diameter of the agitation rotating elements depends on the diameter of the reactor. Generally, the diameter of the rotating element is between 30% and 70% of the diameter of the reactor.

The reactors according to the invention are not microreactors.

The agitation of the reaction medium may be characterized by the Froude number. This parameter is known to those skilled in the art. It is notably defined in the publication: Le génie chimique à l'usage des chimistes [Chemical Engineering for Chemists] by Joseph Lieto, published by Tec & Doc Lavoisier from 1998. The Froude number is calculated according to the following formula: Fr=N²D/g where
g=acceleration of gravity, or 9.81 m/second²
D=diameter of the agitator in metres
N=number of rotations of the agitator in revolutions per second.

Preferably, the Froude number in the first reactor is strictly less than 0.018 and the Froude number in the following reactor(s) is greater than or equal to 0.018. More preferentially, the Froude number in the first reactor is less than 0.010 and the Froude number in the following reactor(s) is greater than 0.018.

The ketone may be introduced at the bottom of the first reactor and the aqueous hydrogen peroxide solution may be introduced by a tube dipping inside this first reactor. The ammonia is introduced into the first reactor in the form dissolved in the aqueous solution containing at least one activator.

The process according to the invention thus makes it possible to reduce the formation of aminoperoxide. In the first reactor, the production of aminoperoxide is low, due to slow agitation. In the following reactors, the high agitation speed enables more rapid consumption of this aminoperoxide to convert it into azine. Thus, the amount of aminoperoxide is reduced, whereas the amount of azine produced is increased.

At the end of the reaction according to the invention, the reaction mixture comprises the azine of the ketone, any unreacted ketone, any activator(s), and any other byproducts or impurities.

### Preparation of the Aqueous Solution

An ammoniacal aqueous solution may be prepared using an absorption column.

The absorption column may be fed with fresh ammonia and with an aqueous solution containing at least one activator.

The absorption column aims to dissolve the gaseous ammonia in the aqueous solution containing at least one activator. The function of the absorption column is to make this mixture of gaseous ammonia and of aqueous solution comprising an activator into a single phase.

At the outlet of the absorption column, an aqueous solution comprising dissolved ammonia in a proportion of between 50% and 100% relative to the saturation of ammonia in pure water at the temperature of the column and comprising at least one activator is obtained. Thus, when an absorption column is used within the process, it is then the temperature of the column that is relevant to the calculation of the ammonia saturation of the aqueous solution.

### Dissolved Ammonia

According to this embodiment, the absorption column aims to dissolve ammonia in the aqueous solution containing activator at a percentage of between 50% and 100% relative to a saturated pure water at the temperature of the column. This solubility of ammonia in the aqueous phase is expressed relative to the amount of water contained in the aqueous phase comprising at least one activator.

Preferably, ammonia is dissolved in the aqueous solution containing activator in a proportion of between 50% and 85% relative to the saturation of ammonia in pure water at the temperature of the column.

The flow rate of fresh ammonia may vary during the process in order to keep constant the solubility of the ammonia in the aqueous solution. At the start of the process, the flow rate of ammonia will have to be sufficient to achieve the desired ammonia solubility. Subsequently, the flow rate will be able to be reduced so as to maintain the desired solubility.

### The Apparatus

The temperature of the absorption column may be between ambient temperature and 70° C., preferably between 20° C. and 50° C., and preferably between 25° C. and 45° C. The pressure of the absorption column may be between atmospheric pressure and up to about 10 bar absolute. Preferably, the reaction is carried out at between 1 and 5 bar absolute.

The column may be a packed or plate distillation column. It is fed with ammonia and with an aqueous solution comprising at least one activator.

It is also possible to completely or partially recover the stream(s) of ammonia generated by the process for preparing the azine.

The aqueous solution containing at least one activator may be a recycled aqueous solution, which originates from the separation step b), which may have completely or partially undergone a regeneration and concentration step.

Preferably, the aqueous solution comprising at least one activator is introduced at the top of the column and the fresh ammonia and/or any recycled ammonia is introduced in countercurrent, preferably at the bottom of the column. The meeting of these streams in countercurrent enables better mixing of the reactants and better absorption of the gaseous ammonia into the aqueous solution.

The ammoniacal aqueous solution comprising at least one activator is then introduced into the first reactor, in which the reaction to form the azine is performed.

Circulation Loop of the Aqueous Solution Saturated with Ammonia

According to one embodiment of the invention, the absorption column may be fed with a stream of the reaction medium of the first reactor of step a). This stream may be withdrawn using a pipe dipping into the reaction medium of the reactor. It is then introduced at the top of the absorption column. Once introduced into the absorption column, this stream originating from the reaction medium of step a) is mixed with the aqueous solution comprising at least one activator, and with fresh ammonia, and optionally with recycled ammonia. At the outlet of the column, the aqueous solution comprising dissolved ammonia in a proportion of between 50% and 100% relative to the saturation of ammonia in pure water at the temperature of the column and comprising at least one activator is sent into the first reactor. This circulation loop between the first reactor and the absorption column makes it possible to constantly maintain a high content of ammonia in the reaction medium of step a). In other words, the stream withdrawn from the reaction medium of step a) will see its concentration of ammonia increased as it passes through the absorption column, before being reinjected into the first reactor.

When the agitation in the first reactor is low, i.e. when the agitation is not sufficient to homogenize the reaction medium, the aqueous phase then tends to be present in a greater concentration at the bottom of the reactor. It is thus advantageous to withdraw the aqueous phase-rich reaction medium from this location to introduce it into the absorption column. The withdrawal pipe is thus positioned preferably in the first third of the height of the liquid phase starting from the bottom of the reactor.

According to one embodiment, the reaction medium of each reactor may be withdrawn and introduced into the absorption column.

According to another embodiment, the reaction medium of a single reactor may be withdrawn and introduced into the absorption column, for example the first reactor or else the last reactor.

According to yet another embodiment, the reaction media of several reactors, but not all the reactors, may be withdrawn and introduced into the absorption column.

### b) Separation Reaction

After the reaction for preparing the azine, the process according to the invention may comprise a step of separating the stream formed on conclusion of the preceding step.

The aqueous phase comprising the activator(s) is separated from the organic phase comprising the azine of the alkyl ketone and any unreacted alkyl ketone by conventional means such as liquid-liquid extraction, distillation, decanting or any combination of these possibilities. Decanting is preferably used.

The organic phase obtained may comprise the azine of the alkyl ketone formed, unreacted alkyl ketone, activator(s), and any other impurities.

### c) Regeneration Reaction

Following the separation step b), the aqueous phase may completely or partially undergo a regeneration and concentration step. This aqueous phase thus regenerated and concentrated may be recycled into the first reactor or else into the ammonia absorption column, when it is present in the process.

During the step of regenerating and concentrating the aqueous phase from the separation step b), a stream of gaseous ammonia may be recycled into the ammonia absorption column, when it is present in the process.

This regeneration step is described in patents EP0399866 and EP0518728.

Following the separation step b), the process may comprise:
a step of washing the organic phase, from the separation step,
a step of hydrolysing the stream obtained from the preceding step to obtain hydrazine hydrate.

### DESCRIPTION OF THE FIGURES

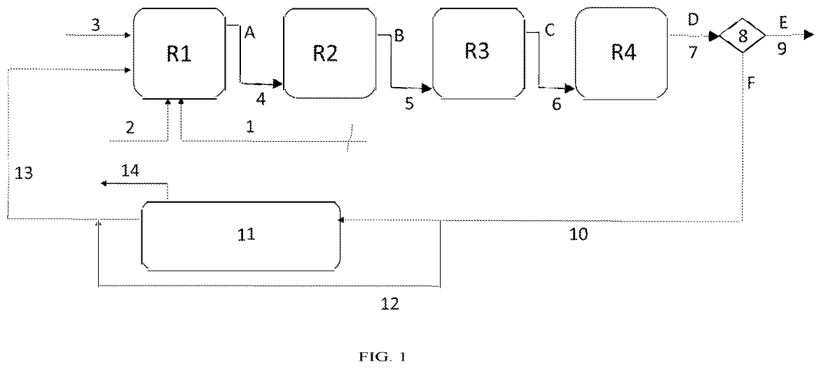

FIG. 1 represents an embodiment of the process according to the invention.

The reaction of ammonia, hydrogen peroxide and the alkyl ketone in the presence of a solution comprising at least one activator to form an azine is carried out in 4 reactors mounted in cascade, denoted R1, R2, R3 and R4.

The reactor R1 is fed with ketone via the line 1. This may be a feed with fresh ketone or else a feed recycling a ketone originating from the process. The reactor R1 is fed with oxygen peroxide via the line 2 and with ammoniacal aqueous solution comprising at least one activator via the line 3.

The lines 4, 5 and 6 bring respectively stream (A), (B) and (C), which come from the reactors R1, R2, R3, to the following reactor, i.e. R2, R3 and R4. The line 7 transports the stream D formed in the reactor R4 to the decanter 8.

The decanter 8 separates the organic phase E and the aqueous phase F. The organic phase E is sent via the line 9 to the subsequent steps of the process. The aqueous phase F is sent to the unit 11 for regeneration and concentration of the aqueous phase F. The line 12 is a line that bypasses the regeneration and concentration unit 11. Depending on the quality of the aqueous phase, it is possible to direct the aqueous phase F to the unit 11 or else to the bypass line 12. It is also possible to send only part of the aqueous phase F to the regeneration and concentration unit 11. The aqueous phase which has undergone the regeneration and concentration step and/or the aqueous phase which has passed via the bypass line 12 is sent to the reactor R1 via the line 13. The regeneration and concentration unit 11 may comprise a purge 14 so as to remove excess water from the circuit.

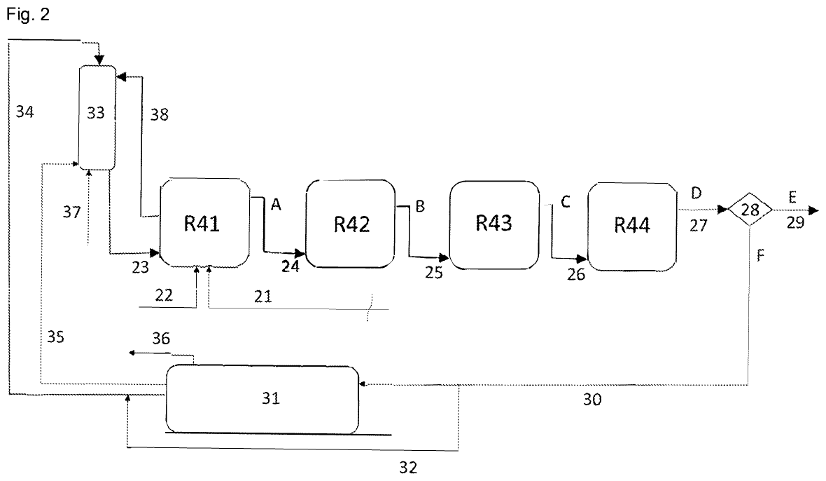

FIG. 2 represents another embodiment of the process according to the invention.

The reaction of ammonia, hydrogen peroxide and the alkyl ketone in the presence of a solution comprising at least one activator to form an azine is carried out in 4 reactors mounted in cascade, denoted R41, R42, R43 and R44.

The reactor R41 is fed with ketone via the line 21. This may be a feed with fresh ketone or else a feed recycling a ketone originating from the process. The reactor R41 is fed with oxygen peroxide via the line 22 and with aqueous solution saturated with ammonia and comprising at least one activator via the line 23.

The lines 24, 25 and 26 bring respectively stream (A), (B) and (C), which come from the reactors R41, R42, R43, to the following reactor, i.e. R42, R43 and R44. The line 27 transports the stream D formed in the reactor R44 to the decanter 28.

The decanter 28 separates the organic phase E and the aqueous phase F. The organic phase E is sent via the line 29 to the subsequent steps of the process. The aqueous phase F is sent to the unit 31 for regeneration and concentration of the aqueous phase F. The line 32 is a line that bypasses the regeneration and concentration unit 31. Depending on the quality of the aqueous phase, it is possible to direct the aqueous phase F to the unit 31 or else to the bypass line 32. It is also possible to send only part of the aqueous phase F to the regeneration and concentration unit 31. The aqueous phase which has undergone the regeneration and concentration step and/or the aqueous phase which has passed via the bypass line 32 is sent to the top of the ammonia absorption column 33 via the line 34. During the regeneration and concentration step, it is also possible to recover ammonia. This ammonia may be recycled via the line 35 to the bottom of the ammonia absorption column 33. The regeneration and concentration unit 31 may comprise a purge 36 so as to remove excess water from the circuit. The ammonia absorption column 33 may also be fed with fresh ammonia via the line 37. Finally, the reaction phase of the reactor R41 is sent to the top of the ammonia absorption column 33, via the line 38.

The line 38, the absorption column 33, the feed line 23 and the reactor R41 form an ammonia recirculation loop.

The examples that follow illustrate the present invention but are not in any way limiting.

### Example

The example compares two processes for preparing azine, one according to the invention and the other comparative.

The reaction step is carried out according to the installation of FIG. 2. The processes use 4 reactors in cascade. Methyl ethyl ketone is used as reactant.

The temperatures of the reactors are as follows: T_{R41}=50° C.; T_{R42}=51° C.; T_{R43}=52.5° C.; T_{R44}=55° C. The reactions are carried out at atmospheric pressure.

The streams of aminoperoxide and of amine that are formed in each reactor are evaluated.

The circulation flow rate in the line 38, i.e. from the reactor R41 to the ammonia absorption column, is 24 t/h. The absorption of ammonia is carried out at a temperature of 30° C.

The flow rates of the reactants arriving in the reactor R41 are given in Table 2 below:

TABLE 2

| | |
|---|---|
| H₂O₂ (100%) | 1401 kg/h |
| Recycled MEK | 6789 kg/h |
| Stream originating from the | 10804 kg/h |
| ammonia absorption column | |

The agitation conditions for each process are reported in Tables 3 and 4 below. The reactors comprise an agitator with a diameter of 1.7 m. The formation of aminoperoxide and the production of azine are evaluated at the outlet of each reactor. The values are given in Tables 3 and 4 below.

TABLE 3

| Ex 1 (counterexample) | R41 | R42 | R43 | R44 |
|---|---|---|---|---|
| Agitation speed (rpm) | 20 | 20 | 20 | 20 |
| Froude number | 0.0190 | 0.0190 | 0.0190 | 0.0190 |
| Aminoperoxide (kg/h) | 407 | 227 | 122 | 59 |
| MEKazine (kg/h) | 2829 | 3964 | 4496 | 4771 |
| Azine yield (%) | 73.2 | 90.4 | 93.0 | 95.6 |

TABLE 4

| Ex 2 (invention) | R41 | R42 | R43 | R44 |
|---|---|---|---|---|
| Agitation speed (rpm) | 10 | 20 | 20 | 20 |
| Froude number | 0.0048 | 0.0190 | 0.0190 | 0.0190 |
| Aminoperoxide (kg/h) | 176 | 146 | 84 | 42 |
| MEKazine (kg/h) | 2945 | 4041 | 4542 | 4797 |
| Azine yield (%) | 77.4 | 89.0 | 93.8 | 95.9 |

These results show that the agitation system as claimed makes it possible to reduce the production of aminoperoxide in each of the reactors. Thus, the difference in agitation applied between the reactor R41 and the reactor R42 has a significant effect on the amount of aminoperoxide produced in these reactors, and this effect is still visible in the reactors R43 and R44. Furthermore, an increase in the yield is also obtained.

1. Process for continuously preparing azine, comprising a step a) of reacting ammonia, hydrogen peroxide and a ketone of formula R₁R₂CO, the groups R₁ and R₂ denoting, independently of one another, a methyl, ethyl, propyl, butyl, pentyl, hexyl, heptyl and octyl group, in the presence of an aqueous solution comprising at least one activator to form an azine,
the reaction being performed in at least 2 reactors arranged in cascade,
the agitation of the first reactor being less than the agitation of the following reactor or each of the following reactors,
the hydrogen peroxide, the ketone and an aqueous solution comprising at least one activator and comprising dissolved ammonia, preferably in a proportion of between 50% and 100% relative to the saturation of ammonia in pure water at the temperature of the aqueous solution, being injected into the first reactor.

2. Process according to claim 1, characterized in that the aqueous solution comprising at least one activator and dissolved ammonia originates from an absorption column.

3. Process according to claim 1, characterized in that the agitation within the first reactor is characterized by a Froude number of strictly less than 0.018 and the agitation of the following reactor(s) is characterized by a Froude number of greater than or equal to 0.018.

4. Process according to claim 1, characterized in that the reaction is carried out in 3, 4 or 5 reactors arranged in cascade.

5. Process according to claim 1, characterized in that the ketone used in step a) is methyl ethyl ketone.

6. Process according to claim 1, characterized in that it comprises, after the reaction step a), a step b) of separating the stream formed on conclusion of the reaction step a).

7. Process according to claim 6, characterized in that it comprises a step c) of regenerating and concentrating the aqueous phase from the separation step b).

8. Process according to claim 7, characterized in that during step c) of regenerating and concentrating the aqueous phase from the separation step b), the aqueous phase thus regenerated and concentrated is recycled into the ammonia absorption column.

9. Process according to claim 7, characterized in that during step c) of regenerating and concentrating the aqueous phase from the separation step b), a gaseous ammonia stream is recycled into the ammonia absorption column.

10. Process according to claim 6, characterized in that it comprises:
a step of washing the organic phase, from the separation step b),
a step of hydrolysing the stream obtained from the preceding step to obtain hydrazine hydrate.
